(19) United States
(12) Patent Application Publication
(10) Pub. No.: US 2002/0120109 A1
(43) Pub. Date: Aug. 29, 2002

(54) Biologically active proteins

(76) Inventors: Gennady P. Samokhin, Lincoln Park, NJ (US); Vladimir P. Torchilin, Charlestown, MA (US); Leonid Z. Iakoubov, Pleasanton, CA (US); Dmitriy A. Mongayt, Swampscott, MA (US)

Correspondence Address: LEE CREWS, PH.D., Fish & Richardson P.C., 225 Franklin Street, Boston, MA, 02110-2804, US

(21) Appl. No.: 09/900,309

(22) Filed: Jul. 6, 2001

Related U.S. Application Data
(60) Provisional application No. 60/216,724, filed on Jul. 7, 2000.

(51) Int. Cl. C12P 21/06; C07K 16/46; C07K 14/00

(52) U.S. Cl. 530/391.1; 530/395; 435/68.1

(57) ABSTRACT

Methods are disclosed for preparing biologically active protein conjugates using negatively charged polymers that protect the protein within the conjugate so that it retains a substantial amount of its biological activity following conjugation (e.g., procedure in which a protein is conjugated to a pharmaceutical agent, a solid support, a reporter molecule, a group carrying a reporter molecule, an acylating agent, a chelating agent, a cross-linking agent, a targeting group, and a ligand and binding group). The invention also includes novel protein conjugates prepared by those methods.

### CLAIM OF PRIORITY

This application claims priority under 35 USC §119(e) to U.S. Patent Application Serial No. 60/216,724, filed on Jul. 7, 2000, the entire contents of which are hereby incorporated by reference.

### TECHNICAL FIELD OF THE INVENTION

This invention relates to new, biologically active protein conjugates and methods for their synthesis.

### BACKGROUND OF THE INVENTION

Proteins, including antibodies, are frequently used in basic research and in clinical settings as diagnostic and therapeutic tools (Yelton, D. E., et al., Ann. Rev. Biochem. 50:657-680 (1961)). Proteins used in these ways have often been modified by adding a modifying agent by conjugation. Modifying agents may act as labels, giving rise to a detectable signal that can easily be followed using standard techniques of detection. For example, adding a modifying agent to a biologically active protein (forming a biologically active protein conjugate) may be useful in following the localization of the protein inside a cell, to analyze its uptake, to follow its fate or to measure its half-life, which otherwise would have been difficult.

An example of a protein that is often modified by conjugation is an antibody (Hiltunen, J. V., Acta Oncol. 32: 831-839 (1993)). Antibodies are often modified with various chemicals or modifying agents to form antibody conjugates for their use in immunofluorescence, in radioimmunoassays, in in vitro assay such as enzyme-linked immunosorbent assay (ELISA), in immunoscintigraphy, or for the targeted delivery of pharmaceutical agents (e.g., a toxin, a drug, or a pro-drug). The necessary prerequisite for these applications is the preservation of at least some of the antibody's biological activity, including its ability to bind to an antigen or antigen analog after modification by conjugation.

A problem encountered during the modification process is that the protein may be affected by modifications in its active site that will change the protein's biological activity including its ability to interact with an effector molecule or with any other interacting molecule. For example, upon conjugation, amino acid residue(s) within an antibody that is crucial for the antibody's biological activity (e.g., an amino acid or a group of amino acids in the active site directly involved in binding or a residue(s) responsible for sustaining active site conformation) may be so reactive with a modifying agent that the antibody loses its biological activity (Torchilin, V. P., et al., Biochem. Biophys. Acta 567:1-11 (1979)). Under these circumstance, even the treatment of the antibody with low concentration of the modifying agent leads to the loss of its biological activity.

When it is not feasible to employ an alternative modifying agent (e.g., one that reacts only with non-crucial amino acid residue(s)), the antibody's active site may be protected by exposing it to an antigen or antigen analog (epitope) that is recognized by the antibody and that temporarily masks the residue(s) crucial for biological activity (Ramjeeshingh, M., et al., J. Immunol. Methods 133:159-167 (1990)). This process requires one to know the antigen or antigen analog (epitope) that is usually recognized by the antibody and to have it in sufficient quantity and in pure form to protect the residue(s) crucial for the biological activity upon conjugation. Furthermore, for this process to succeed, it is preferable that the masking antigen or antigen analog (epitope) would not interfere with the antibody modification. It is preferable also that the masking antigen or antigen analog be removed from the reaction mixture after the modification is completed in order to restore normal properties of the protein.

### SUMMARY OF THE INVENTION

The present invention features new protein conjugates and methods of making these protein conjugates so that the proteins within them retain a substantial amount of biological activity following modification by conjugation forming a biologically active protein conjugate. For example, a protein within a protein conjugate of the invention may retain at least 50% (e.g. 60%, 70%, 80%, 90%, 95%, or 100%) of its biological activity. One of ordinary skill in the art will understand, however, that in some cases, a biological activity of 50% or less can be sufficient.

In a first aspect, the present invention relates to a method for preparing a biologically active protein conjugate. The method includes: combining (e.g. mixing) a biologically active protein moiety with a protective group under conditions that allow the protective group to removably (e.g., reversibly) bind to the protein to provide a protected protein; modifying the protected protein by the addition of a conjugate or a modifying agent moiety (e.g. a pharmaceutical agent, a solid support, a reporter group, or a targeting group); and removing the protective group to provide a biologically active protein conjugate that includes a biologically active protein moiety and the modifying agent moiety.

In a second aspect, the present invention relates to a biologically active protein conjugate comprising a first moiety and a second moiety, wherein the first moiety is a biologically active protein and wherein the second moiety is a pharmaceutical agent, a solid support, a reporter molecule, or a targeting group.

In accordance with the present invention, the biologically active conjugate may also include a protective group.

In a third aspect, the present invention relates to a method for preparing a protected protein, the method comprising combining (e.g. mixing) a biologically active protein to an anionic saccharide substance (e.g., anionic polysaccharides, anionic oligosaccharides or mixtures thereof), so as to removably (e.g., reversibly) bind a protective group to the biologically active protein and obtain a protected protein that includes the biologically active protein and the protective group.

In a fourth aspect, the present invention relates to a method of preparing a biologically active protein conjugate. The method includes: combining (e.g. mixing) a biologically active protein with a protective group selected from the group consisting of anionic polysaccharides, anionic oligosaccharides, and mixtures thereof to provide a protected protein under conditions that allow the protective group to removably (e.g., reversibly) bind to the protein, and modifying the protected protein by the addition of a conjugate or modifying agent (e.g., a pharmaceutical agent, a solid support, a reporter molecule, a group carrying a reporter molecule, a chelating agent, an acylating agent, a cross-linking agent, and a targeting group).

In accordance with the present invention, the methods described in the fourth aspect of the invention may also include the step of removing the protective group to provide a biologically active protein conjugate that includes a biologically active protein and a modifying agent or conjugate.

In a fifth aspect, the present invention relates to a protected biologically active protein that includes a biologically active protein associated with a protective group such as an anionic polysaccharide, an anionic oligosaccharide, or mixtures thereof.

In accordance with the present invention, the term “protected biologically active protein” refers to a biologically active protein protected with a protective group, wherein the biologically active protein recovers at least part of its biological activity after removal of the protective group.

In a sixth aspect, the present invention relates to a biologically active protein conjugate that includes a first moiety and a second moiety, the first moiety being a protected biologically active protein and the second moiety being a pharmaceutical agent, a solid support, a reporter molecule, a group carrying a reporter molecule, a chelating agent, an acylating agent, a cross-linking agent, or a targeting group. The protected biologically active protein can be associated with a protective group such as an anionic polysaccharide, an anionic oligosaccharide or mixtures thereof.

In accordance with the present invention, the biologically active protein may be, for example, an antibody, an enzyme, a receptor, a cytokine, a chemokine, a growth factor, a hormone, a transcription factor, a peptide (e.g., a protein fragment), a peptide analog or any protein that may specifically bind to a protein, glycoprotein, nucleic acid or mixtures thereof.

In accordance with the present invention, a protective group may be a negatively charged polymer and may, for example, include one or more of the following: carboxylates, sulfates, sulfonates, phosphonates, phosphates, and the like. The negatively charged polymer may be a natural or synthetic polymer such as carboxymethyl-cellulose, carboxymethyl-starch and carboxymethyl-dextran, or an anionic saccharide including anionic polysaccharides (e.g. anionic dextrans), anionic oligosaccharides and mixtures thereof. Anionic polysaccharides and anionic oligosaccharides include for example, dextran sulfate (DexSO₄) and heparin (Hep) as well as pectin and xanthan gum. Polymers (including branched and unbranched polymers) of greatly differing size may be effective as a protective group in the present invention (e.g., dextran sulfate having a molecular mass of either 10,000 or 500,000 dalton (Da) may be used). Protective groups are of course chosen based on their ability to protect a desired protein.

In accordance with the present invention, the modifying agent moiety may be, for example, a pharmaceutical agent (e.g., a toxin, a drug, and a pro-drug), a solid support (e.g., the matrix of an affinity column, a carbohydrate, a liposome, a lipid, a microparticle, a microcapsule, a microemulsion, or colloidal gold), a targeting group (e.g., antibody fragments, hormones, or lectins), a reporter molecule or a group that may carry a reporter molecule. The reporter molecule may be, for example, a fluorophore, a chromophore, or dye (e.g., rhodamine, fluoroscein, or green fluorescent protein) or any other agent or label that gives rise to a detectable signal, either by acting alone or following a biochemical reaction (e.g., horseradish peroxidase, alkaline phosphatase, and beta-galactosidase). A group that can carry a reporter molecule may be, for example, diethylenetriaminepentaacetic acid (DTPA). Diethylenetriaminepentaacetic acid anhydride (DTPA-A) is an acylating agent (i.e., a compound that can modify an amino group) that also acts as a chelating agent that is able to bind to heavy metal ions including radioisotopes (e.g. Isotope 111 of Indium (¹¹¹In)) that act as reporter molecules.

In accordance with the present invention, the conditions that allow the protective group to be removably bound to the protein are selected keeping in mind the purpose of the protecting group relative to the protein and may reflect conditions such as those given in the examples below.

In accordance with the present invention, conditions that may allow the protective group to be removed from the protein may be, for example, those in which a high ionic strength solution is used to dissociate the biologically active protein from the protective group. Such high ionic strength solution may include the use of 1 M NaCl (e.g. aqueous sale solution including for example a salt such as sodium chloride, potassium chloride, sodium acetate, and the like). The concentration or molarity of the solution is, of course, chosen for its efficiency to allow removal of the protective group from the protein.

In accordance with the present invention, conditions that allow the protective group to removably bind to the protein may be, for example, by way of using a high ionic strength solution to dissociate the biologically active protein from the protective group. An example of a solution of high ionic strength is 1M NaCl.

The protein conjugates of the present invention include those synthesized by the methods described herein, which allow the protein within the protein conjugate to retain its biological activity after being subjected to a given conjugation reaction. The methods of the invention may be carried out by combining biologically active proteins with protective groups, giving rise to a protected protein that may be conjugated to a modifying agent. The protective group may mimic a molecule or part of a molecule to which the biologically active protein would bind, thus protecting the protein's active site upon conjugation.

Protective groups of the present invention may mimic the antigen or antigen analog that is usually recognized by an antibody without being the antigen or antigen analog itself. Thus, in case the biologically active protein is an antibody, the protective groups may mimic the antigen or antigen analog. In the event the biologically active protein is an enzyme, the protective group may mimic the substrate of the enzyme. In the event the biologically active protein is a transcription factor, the protective group may mimic DNA and/or other proteins that are usually recognized by the transcription factor without being DNA and/or other proteins themselves. In the event the biologically active protein is a receptor, the protective group may mimic the ligand that is usually recognized by the receptor without being the ligand itself. In the event the biologically active protein is a cytokine, a chemokine, a growth factor or a hormone, the protective group may mimic a desired receptor that is usually recognized by the cytokine, chemokine, growth factor or hormone without being the receptor itself. The interaction between the active site of a protein (e.g. the antigen binding site, substrate binding site) and the protective group enables the protein to keep at least part of its biological activity prior to or during subsequent conjugation reactions.

As described herein, the protective group may be removed using high ionic strength solutions and the biologically active protein conjugate may be used in a variety of ways including immunofluorescence, radioimmunoassays, enzyme-linked immunosorbent assays, in immunoscintigraphy, or for the targeted delivery of pharmaceutical agents (e.g., a toxin, a drug, or a pro-drug).

By modifying a biologically active protein by conjugation to form a biologically active protein conjugate, one can, for example, study the fate of the biologically active protein conjugate in vivo. For example, a biologically active protein conjugate containing the 2C5 monoclonal antibody (also named herein antinuclear autoantibody or ANA) as the biologically active protein moiety and a group carrying a reporter molecule as the second moiety may be used to determine the circulating half-life of the 2C5 monoclonal antibody conjugate and its distribution among the various organs of the body.

Novel biologically active protein conjugates of the present invention include ANA (i.e. 2C5 monoclonal antibody) conjugated to the chelating agent DTPA-A (e.g. a 2C5-DTPA-conjugated monoclonal antibody) and to the radioisotope ¹¹¹In.

The invention has numerous advantages. Previously available methods require one to know the interacting molecule that is recognized by the protein and to have it in sufficient quantity and in pure form to protect the residue(s) crucial for the biological activity prior or during conjugation. This requirement is obviated by the present methods.

Another important advantage of the method described herein is that the biologically active protein moiety (e.g. the antibody) within the biologically active protein conjugate remains biologically active following conjugation, since the method allows the protein's active site to be protected from modification. When previously available methods are used, even low concentrations of modifying agents result in the loss of a protein's biological activity when the active site is not protected upon conjugation. As will become apparent from the information presented here, the present method enables the protein to retain at least part of its biological activity and obviates use of the antigen or antigen-analogs usually recognized by the antibody for protection of the active site upon conjugation. Other features and advantages will be apparent from the following detailed description and from the claims.

Unless otherwise defined, all technical and scientific terms used herein have the same meaning as commonly understood by one of ordinary skill in the art to which this invention belongs. Although methods and materials similar or equivalent to those described herein may be used in the practice or testing of the present invention, suitable methods and materials are described below. All publications, patent applications, patents, and other references mentioned herein are incorporated by reference. The materials, methods, and examples serve to illustrate, not limit, the invention.

### BRIEF DESCRIPTION OF THE DRAWINGS

FIG. 1 is a line graph depicting the protective effect of dextran sulfate on the biological activity of 2C5 monoclonal antibody upon conjugation with DTPA-A. The biological activity of the 2C5 DTPA-conjugated antibody is illustrated by its ability to bind to nucleohistone (NH) preparation adsorbed to poly-L-lysine coated plates and is measured by ELISA. Results of binding of the 2C5 monoclonal antibody to the nucleohistone-coated plates, illustrated by the optical density measured at 630 nm after the enzymatic reaction has proceeded, are expressed as a function of the 2C5 monoclonal antibody concentration. Open circles represent the non-modified 2C5 monoclonal antibody control. Solid circles represent the 2C5 monoclonal antibody conjugated with DTPA-A without protection by dextran sulfate. Open squares represent the 2C5 monoclonal antibody protected with dextran sulfate 10,000 Da without any conjugation by DTPA-A. Solid squares represent the 2C5 monoclonal antibody protected with dextran sulfate 10,000 Da and conjugated with DTPA-A. Open triangles represent the 2C5 monoclonal antibody protected with dextran sulfate 10,000 Da and incubated with NaCl. Solid triangles represent the 2C5 monoclonal antibody protected with dextran sulfate 10,000 Da and conjugated with DTPA-A in the presence of NaCl.

FIG. 2 is a line graph depicting the protective effect of dextran sulfate on the biological activity of 2C5 monoclonal antibody upon conjugation with 3-(2-pyridyldithio) propionic acid N-hydroxysuccinimide ester (SPDP). The biological activity of the 2C5 SPDP-conjugated antibody is illustrated by its ability to bind to nucleohistone (NH) preparation adsorbed to poly-L-lysine coated plates and is measured by ELISA. Results of binding of the 2C5 monoclonal antibody to the nucleohistone-coated plates, illustrated by the optical density measured at 630 nm after the enzymatic reaction has proceeded, are expressed as a function of 2C5 monoclonal antibody concentration. Open circles represent the non-modified 2C5 monoclonal antibody control. Solid circles represent the 2C5 monoclonal antibody conjugated with SPDP without protection by dextran sulfate. Open squares represent the 2C5 monoclonal antibody protected with dextran sulfate 10,000 Da, without conjugation by SPDP. Solid squares represent the 2C5 monoclonal antibody protected with dextran sulfate 10,000 Da and conjugated with SPDP.

FIG. 3 is a line graph depicting the protective effect of heparin on the biological activity of 2C5 monoclonal antibody upon conjugation with DTPA-A. The biological activity of the 2C5 DTPA-conjugated antibody is illustrated by its ability to bind to nucleohistone (NH) preparation adsorbed to poly-L-lysine coated plates and is measured by ELISA. Results of binding of the 2C5 monoclonal antibody to the nucleohistone-coated plates, illustrated by the optical density measured at 630 nm after the enzymatic reaction has proceeded, are expressed as a function of 2C5 monoclonal antibody concentration. Open circles represent the non-modified 2C5 monoclonal antibody control. Closed circles represent the 2C5 monoclonal antibody conjugated with DTPA-A without protection by heparin. Open triangles represent the 2C5 monoclonal antibody protected with heparin without any conjugation by DTPA-A. Solid triangles represent the 2C5 monoclonal antibody protected with heparin and conjugated with DTPA-A.

FIG. 4 is a line graph depicting the biological activity of the 2C5-DTPA-conjugated monoclonal antibody labeled with ¹¹¹In, generated by protection of the active site with dextran sulfate (10 000 Da). The ability of 2C5 ¹¹¹In DTPA-conjugated antibody to bind to nucleohistone preparation adsorbed to poly-L-lysine coated plates is measured by radioactivity remaining bound to plate after adsorption of the 2C5 ¹¹¹In DTPA-conjugated antibody and subsequent washing. Results of binding of the 2C5 ¹¹¹In DTPA-conjugated antibody to the antigen-coated plates are expressed as a function of 2C5 ¹¹¹In DTPA-conjugated antibody concentration. Solid circles represent the 2C5 ¹¹¹In DTPA-conjugated antibody bound to nucleohistone preparation adsorbed to poly-L-lysine coated plates. Open circles represent the negative control of 2C5¹ ¹¹¹In DTPA-conjugated antibody bound to non-coated plates.

FIG. 5 is a line graph depicting the presence of the 2C5 ¹¹¹In DTPA-conjugated antibody generated by protection of the antigen-binding site with dextran sulfate (10,000 Da) in mouse blood as a function of time following injection.

FIG. 6 is a line graph depicting the presence of the 2C5 ¹¹¹In DTPA-conjugated antibody generated by protection of the antigen-binding site with dextran sulfate (10,000 Da) in mouse liver as a function of time following injection.

FIG. 7 is a line graph depicting the presence of the 2C5 ¹¹¹In DTPA-conjugated antibody, generated by protection of the antigen-binding site with dextran sulfate (10,000 Da) in mouse kidney as a function of time following injection.

FIG. 8 is a line graph depicting the presence of the 2C5 ¹¹¹In DTPA-conjugated antibody generated by protection of the antigen-binding site with dextran sulfate (10,000 Da) in mouse spleen as a function of time following injection.

FIG. 9 is a line graph depicting the presence of the 2C5 ¹¹¹In DTPA-conjugated antibody generated by protection of the antigen-binding site with dextran sulfate (10,000 Da) in mouse lungs as a function of time following injection.

### DETAILED DESCRIPTION

The methods described herein are based on the observation that conventional methods of modifying an antibody by conjugation may decrease the biological activity of the antibody. The method described herein enables the protein to keep some of its biological activity and obviates the use of the antigen or antigen-analogs usually recognized by the antibody for protection of the active site upon conjugation. The invention described herein is not limited to proteins that lose their biological activity by a particular mechanism.

As used herein the term “polymer” refers to a large molecule formed by the union of monomers (e.g., identical monomers) and includes natural polymers, synthetic polymers, branched polymers and linear polymers.

As used herein, the term “antibody” refers to either monoclonal antibody, polyclonal antibody, humanized antibody, antibody fragments including Fc, F(ab)2, F(ab)2′ and Fab and the like.

As used herein, the term “biological activity” (or analogous terms) of a protein refers to the activity that is usually carried out by such protein and includes the enzymatic activity of a protein as well as its effector function and its ability to bind other molecules important for its activity (e.g., antigen, antigen analog or epitope in the event the protein is an antibody, substrates in the event the protein is an enzyme, DNA and/or other proteins in the event the protein is a transcription factor, a ligand in the event the protein is a receptor, a receptor in the event the biologically active protein is a cytokine, a chemokine, a growth factor and an hormone).

As used herein, the term “epitope” includes linear and conformational epitope and refers to the group of atoms that are recognized by an antibody's antigen binding site.

As used herein, the term(s) “active site” or “biologically active site” refer to a region of a protein that is responsible for its biological activity and includes an antigen binding site and a substrate binding site.

As used herein the term “modifying agent” refers to a molecule or group of molecule that can be added (covalently or noncovalently) to a protein and includes pharmaceutical agents, solid supports, reporter molecule, groups carrying a reporter molecule, acylating agents, chelating agents, cross-linking agent, targeting groups, or ligand and binding groups.

As used herein, the terms “protein conjugate” and “antibody conjugate” refer to a protein or antibody that as been modified by the addition of a desired agent (e.g., modifying agents) and include a 2C5-DTPA-conjugated antibody and a 2C5 ¹¹¹In DTPA-conjugated antibody.

As used herein, the term “reporter molecule(s)” refers to molecule(s) that give rise to a detectable signal and include fluorescent molecules (e.g., rhodamine and fluoroscein), enzymes (e.g., horseradish peroxidase), dyes, radioactive atoms and isotopes (e.g., indium, iodine and technetium), and superparamagnetic and paramagnetic agents (e.g., gadolinium and iron, and manganese).

As used herein, the term “chelating agent” refers to a compound capable of forming chemical bonds with metal ion through two or more of its atoms. Chelating agents include DTPA-A and Ethylenediaminetetraacetic acid (EDTA).

As used herein, the term “cross-linking agents” refers to compounds able to link two or more entities. An exemplary cross-linking agent is SPDP.

As used herein, the term “solid support” includes liposomes, colloidal gold, microparticles, and microcapsules.

As used herein, the terms “ligands and binding groups” include one of a pair of such ligand/binding groups such as biotin and avidin or biotin and streptavidin.

Proteins encompassed by the present invention include any protein with a biological activity. Specifically and by way of example only, encompassed by the present invention are antibodies, enzymes, cytokines, chemokines, growth factors, hormones, receptors and transcription factors or any protein interacting with another molecule.

More specifically, and by way of example only, the present invention relates to a modified 2C5 monoclonal antibody. It has been recently shown that certain naturally occurring nonpathogenic ANAs (e.g., 2C5 monoclonal antibody) may selectively recognize and kill a broad variety of cancer cells both in vitro and in vivo (Iakoubov, L., et al., Immunol. Lett. 47:147-149 (1995), Iakoubov, L., et al., Oncol. Res., 9:489-446 (1997)). The 2C5 monoclonal antibody possesses specificity for nucleosome, meaning that this antibody is able to bind nucleosomes (Iakoubov, L. et al. Oncol. Res. 9: 439-446 (1997)).

Examples described herein indicate that the biological activity of the 2C5 monoclonal antibody measured by its ability to bind to a nucleohistone preparation adsorbed to poly-L-lysine coated plates in ELISA is decreased by up to 97% when it is modified by a modifying agent such as DTPA-A using a conventional method of conjugation. Moreover, the 2C5 monoclonal antibody loses its biological activity even when the concentration of the chelating agent is only 100 μM (i.e., when the initial molar ratio of 2C5:DTPA-A is 1:20).

Highly reactive amino groups within the protein's active site are not only crucial for the protein's biological activity, but also may adversely be affected by one or more of the compounds that are used in the conjugation process (e.g., pharmaceutical agents, solid supports or substrates, reporter molecules, groups carrying a reporter molecule, acylating agents, chelating agents, cross-linking agents, targeting groups, or ligand and binding groups).

Proteins that may be successfully be conjugated by the methods described herein include those that bind negatively charged molecules, such as DNA (i.e., anti-DNA antibodies). Other biologically active proteins are known to bind to negatively charged molecules. Examples of proteins having affinity for negatively charged molecules include nucleases (e.g., DNAse and RNAse), DNA synthases, DNA kinases, transcription factors, and enzymes whose substrates are negatively charged (e.g., heparinase) are encompassed by the present invention.

One specific example of a protein that binds a negatively charged molecule is the 2C5 monoclonal antibody, which binds to nucleosomes.

The active site of 2C5 may be protected from modification by including charged polymers such as anionic polysaccharides and anionic oligosaccharides as a protective group prior to or during the conjugation process (i.e., in the course of conjugating the 2C5 monoclonal antibody to a modifying agent as described herein).

### EXAMPLES

Conventional modification of antibodies of proteins may be made by first reacting them with a modifying agent such as DTPA-A, which has the ability to chelate metallic ions, followed by the addition of the metallic ion itself such ¹¹¹In, gadolinium (Gd) or manganese (Mn) (which act as reporter molecules in standard techniques of detection).

The examples below demonstrate that when conventional methods are used to modify an antibody protein, such as the 2C5 monoclonal antibody, with a modifying agent such as DTPA-A, the biological activity of the antibody is reduced by up to 97%. This reduction is observed even when the concentration of the modifying agent is only 100 μM (i.e., when the initial molar ratio of 2C5 to DTPA-A was 1:20).

The examples also show that incubation of the antibody (i.e. the 2C5 monoclonal antibody), with dextran sulfate alone or with heparin alone did not lead in the loss of the biological activity of the antibody. Furthermore, modification of the 2C5 monoclonal antibody with DTPA-A or SPDP in the presence of dextran sulfate or heparin results in a protein conjugate that remains biologically active. These results indicate that dextran sulfate and heparin act by protecting the biologically active site of the 2C5 monoclonal antibody from modification by DTPA-A or SPDP. Thus, dextran sulfate and heparin seem to mimic the antigen that is usually recognized by the 2C5 monoclonal antibody. The most probable explanation for the mimicking ability of dextran sulfate and heparin, which are negatively charged polymers (anionic polysaccharides and anionic oligosaccharides respectively), is that the interaction between the 2C5 monoclonal antibody and the antigen may be also through electrostatic forces.

Results described herein are consistent with the hypothesis that DTPA-A-sensitive amino groups in the antigen binding site of the 2C5 monoclonal antibody interact through electrostatic forces with nucleosomes and are required for successful interaction between the antigen or the antigen analog and the antibody. To test this hypothesis further, antibodies were conjugated with a modifying agent in the presence of dextran sulfate at high ionic strength (i.e., in the presence of 1M NaCl).

The presence of 1M NaCl during the modification reaction drastically reduced the ability of dextran sulfate to protect the 2C5 monoclonal antibody from losing its biological activity following modification, as evidenced by up to 90% loss in the antibody's binding activity to a nucleohistone preparation in such experimental conditions. However, incubation of the 2C5 monoclonal antibody at high ionic strength (i.e., in the presence of 1M NaCl), without modification by conjugation did not affect the antibody's biological activity, showing that 1M NaCl by itself is not responsible for the loss of the 2C5 monoclonal antibody's biological activity.

### Example 1

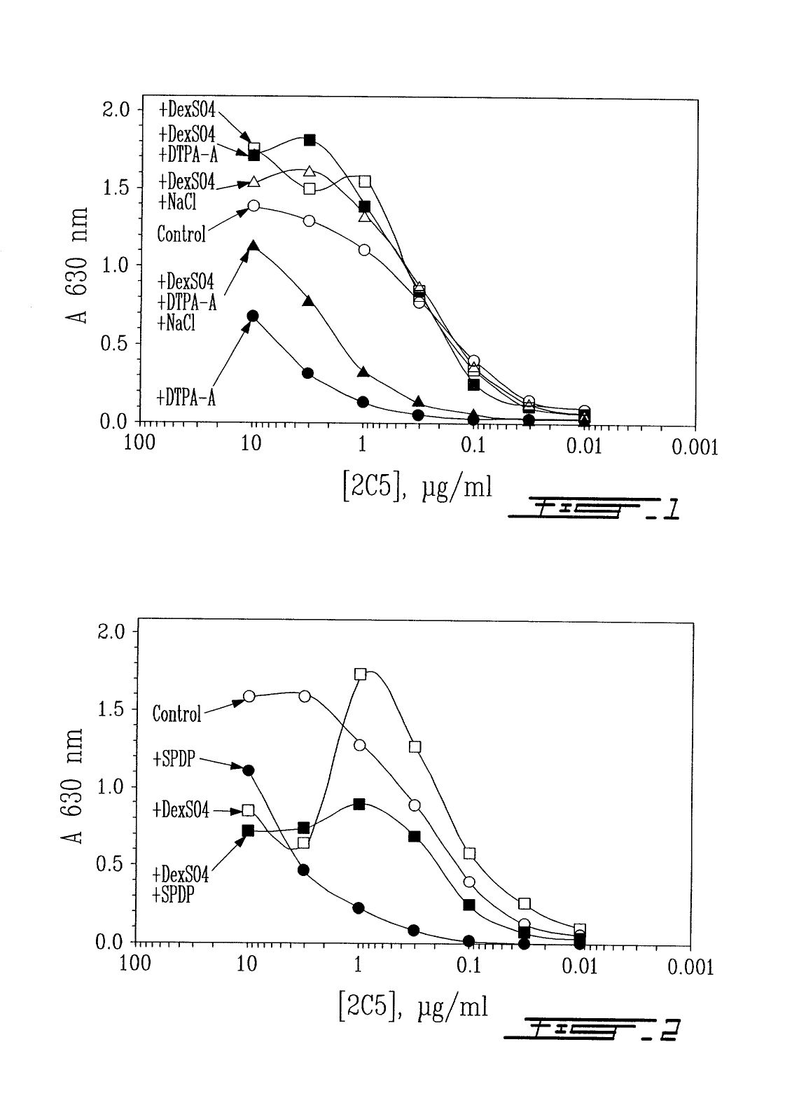

### 2C5 Monoclonal Antibody Modification with DTPA-A in the Presence of Dextran Sulfate (10,000 Da) and/or High Ionic Strength. (Data Are Presented in FIG. 1)

Reagents.

The origin of the mouse hybridoma producing the 2C5 monoclonal antibody was described earlier (Iakoubov, L. Z., et al., Oncol. Res. 9: 489-446 (1997)). Hybridoma was grown as an ascite, and the 2C5 monoclonal antibody was purified by ammonium sulfate precipitation (at 50% saturation) and subsequent ion-exchange chromatography on DEAE-Toyopearl 650M (Sigma, St. Louis, Mo.). Diethylenetriaminepentaacetic acid anhydride (DTPA-A), 3-(2-pyridyldithio) propionic acid N-hydroxysuccinimide ester (SPDP), dextran sulfate (DexSO₄), molecular mass of 10,000 and 500,000 Da, heparin (H-3149), dimethyl sulfoxide (DMSO), salts and buffers were from Sigma (St. Louis, Mo.). 96-well polyvinylchloride microplates were from Costar (Cambridge, Mass., Cat. No. 2596). Anti-mouse IgG horseradish peroxidase-conjugate and HEPES (N-[2-hydroxyethyl]piperazine-N′-[2-ethanesulfonic acid])were from ICN (Costa Mesa, CA). K-Blue peroxidase substrate was from Neogen (Louisville, Ky.). HiTrap recombinant protein A (r-pA) column (1 ml) was from Amersham Pharmacia Biotech (Piscataway, N.J.). Nucleohistone was from Worthington (Lakewood, N.J.).

Antibody Activity Determination.

The antibody binding to commercial nucleohistone preparation adsorbed to poly-L-lysine-coated plates has been done by enzyme-linked immunosorbent assay (ELISA) (Iakoubov, L. Z., et al., Oncol. Res. 9:489-446 (1997)). To assess the effect of modification on antibody activity, we used the 2C5 monoclonal antibody concentration providing 20% or 30% maximal response in each experiment, using non-modified 2C5 monoclonal antibody as 100% reference point (i.e. biological activity given for non-modified 2C5 monoclonal antibody is set at 100%).

Antibody Modification.

To 8.5 μl of 0.94 mg/ml 2C5 monoclonal antibody in 10 mM HEPES, pH 7.5 1 μl of DexSO₄ (10 mg/ml in the same buffer) or buffer, and 0.5 μl of DTPA-A (0.6-20 mM in DMSO, freshly prepared) was sequentially added and the mixture was incubated for 1 hr at room temperature. Those experimental conditions correspond to results represented by solid squares in FIG. 1. The 2C5 monoclonal antibody modification was also performed in other conditions in order to have proper controls for interpretation of results. The results for the non-modified 2C5 monoclonal antibody control are represented by open circles of FIG. 1. This non-modified 2C5 monoclonal antibody control was taken through exactly the same modification procedures with the exception of addition of DTPA-A. Results of incubation of the 2C5 monoclonal antibody with dextran sulfate alone are represented by open squares of FIG. 1. Results of conjugation of the 2C5 monoclonal antibody by DTPA-A in the presence of dextran sulfate and NaCl are represented by solid triangles of FIG. 1. Results of conjugation of the 2C5 monoclonal antibody with DTPA-A in the absence of a protective agent are represented by solid circles of FIG. 1. Results of incubation of the 2C5 monoclonal antibody with dextran sulfate and NaCl are represented by open triangles of FIG. 1. Unless otherwise mentioned, the other parameters of the reaction (i.e., temperature, pH, molarity of buffers, time of incubation etc.) were exactly the same for each point of the graph.

Antibody activity was determined by ELISA. Briefly, after performing modifications of the antibody according to the conditions specified herein, the modified-antibody (or controls) was applied to plates coated with poly-L-lysine-and nucleohistone preparation. Serial dilutions of the antibody (or controls) were performed inside the plate in order to obtain the following concentration of antibody: 0.010, 0.032, 0.100, 0.316, 1.000, 3.162, 10.000 μg/ml. An anti-mouse IgG horseradish peroxidase conjugated antibody was added. The plate was washed and the peroxidase substrate (K-blue) was added. The optical density in each wells of the plate was measured at 630 nm following the enzymatic reaction. Raw data of the ELISA are presented in Table 1, below. Row 1 of Table 1 represents data obtained for the non-modified 2C5 monoclonal antibody control. Row 2 of Table 1 represents data obtained for the 2C5 monoclonal antibody conjugated with DTPA-A in the absence of protecting agent. Row 3 of table 1 represents data obtained for the 2C5 monoclonal antibody incubated with dextran sulfate alone. Row 4 of Table 1 represents data obtained for the 2C5 monoclonal antibody protected with dextran sulfate and conjugated with DTPA-A. Row 5 of Table 1 represents data obtained for the 2C5 monoclonal antibody incubated with dextran sulfate and 1M NaCl. Row 6 of Table 1 represents data obtained for the 2C5 monoclonal antibody protected with dextran sulfate and conjugated with DTPA-A in the presence of 1M NaCl.

TABLE 1

| | ROW NUMBER | | | | | | [2C5] |
|---|---|---|---|---|---|---|---|
| LANE | 1 | 2 | 3 | 4 | 5 | 6 | (μg/ml) |
| A | 1.395 | 0.679 | 1.762 | 1.718 | 1.554 | 1.142 | 10.000 |
| B | 1.297 | 0.318 | 1.506 | 1.821 | 1.62 | 0.780 | 3.162 |
| C | 1.107 | 0.131 | 1.555 | 1.397 | 1.337 | 0.335 | 1.000 |
| D | 0.777 | 0.046 | 0.820 | 0.845 | 0.876 | 0.142 | 0.316 |
| E | 0.409 | 0.034 | 0.347 | 0.264 | 0.369 | 0.053 | 0.100 |
| F | 0.148 | 0.027 | 0.102 | 0.103 | 0.129 | 0.029 | 0.032 |
| G | 0.086 | 0.027 | 0.060 | 0.051 | 0.050 | 0.022 | 0.010 |

When the 2C5 monoclonal antibody was modified with DTPA-A without protection of the active site, the antibody lost up to 97% of its biological activity following conjugation (FIG. 1, solid circles compared with the open circles). In this particular experiment the concentration of DTPA-A was low (i.e. 100 μM, the initial molar ratio 2C5:DTPA-A was 1:20). However, even this low concentration of DTPA-A resulted in the lost of the biological activity of the 2C5 monoclonal antibody conjugate.

The impact of dextran sulfate (a negatively charged polymer essentially inert in terms of acylation reaction) on the biological activity of the 2C5 monoclonal antibody was tested. An experiment was performed by incubating the 2C5 monoclonal antibody with dextran sulfate (10,000 Da) alone. Results illustrated in FIG. 1 (FIG. 1, open squares) indicate that dextran sulfate itself does not lead to the loss of the 2C5 monoclonal antibody's activity when compared to control (FIG. 1, open circles). Dextran sulfate, was thus tested as a protecting agent, during the modification process with DTPA-A. Result of this experiment is illustrated in FIG. 1 (solid squares). These results indicate that treatment of the 2C5 monoclonal antibody with DTPA-A in the presence of dextran sulfate results in the preservation of its biological activity (FIG. 1, solid squares compared with open circles).

If amino groups in the 2C5 monoclonal antibody antigen binding site provides crucial electrostatic interaction with nucleosome, then it is reasonable to suggest that the interaction between the 2C5 monoclonal antibody and the antigen (and the interaction of the 2C5 monoclonal antibody with dextran sulfate as well) it is suspected that this interaction will depend on the ionic strength of the reaction medium. Thus, to further test this hypothesis, antibody modification was performed in the presence of dextran sulfate at high ionic strength (i.e. in the presence of 1M NaCl). As shown in FIG. 1, incubation of the 2C5 monoclonal antibody in the presence of NaCl and dextran sulfate does not affect the antibody's biological activity (FIG. 1, open triangles) compared to control (FIG. 1, open circles). However, the presence of 1 M NaCl during the modification reaction (i.e., in the presence of dextran sulfate, DTPA-A and NaCl) drastically reduces the protecting ability of dextran sulfate (FIG. 1, solid triangles) to protect the 2C5 monoclonal antibody from losing its biological activity during the modification process. Results of this experiment indicate that up to 90% of the biological activity of the 2C5 monoclonal antibody is lost if the modification is performed in the presence of NaCl even if dextran sulfate is added as a protective group in the reaction (FIG. 1, solid triangle compared to control: open circles).

These results indicate that the high ionic strength provided by NaCl interfere with the protecting effect that has been observed with dextran sulfate. An explanation for this phenomenon is that binding of dextran sulfate to the active site of the 2C5 monoclonal antibody is disrupted when NaCl is present. These results also suggest that NaCl may be used to dissociate dextran sulfate from the 2C5 monoclonal antibody after the conjugation reaction has proceeded.

### Example 2

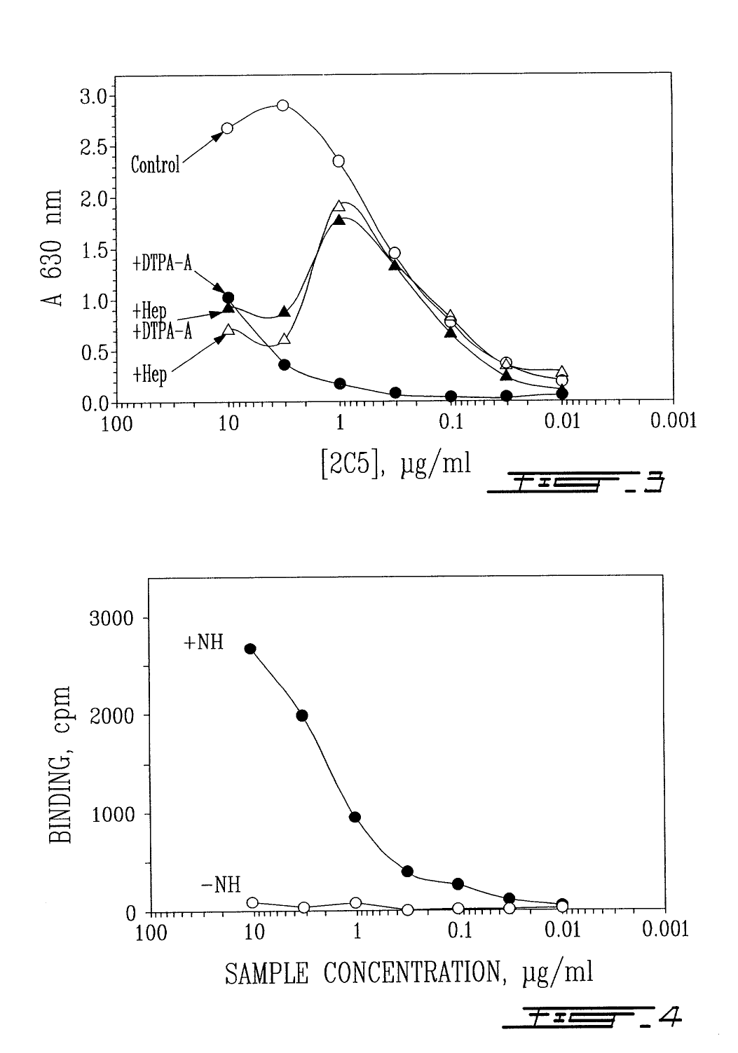

### 2C5 Monoclonal Antibody Modification with SPDP in the Presence of Dextran Sulfate (10,000 Da). (Data are Presented in FIG. 2)

Reagents.

The origin of the mouse hybridoma producing the 2C5 monoclonal antibody was described earlier (Iakoubov, L. Z., et al., Oncol. Res. 9: 489-446 (1997)). Hybridoma was grown as an ascite and the 2C5 monoclonal antibody was purified by ammonium sulfate precipitation (at 50% saturation) and subsequent ion-exchange chromatography on DEAE-Toyopearl 650M (Sigma, St. Louis, Mo.). Diethylenetriaminepentaacetic acid anhydride (DTPA-A), 3-(2-pyridyldithio)propionic acid N-hydroxysuccinimide ester (SPDP), dextran sulfate (DexSO₄), molecular mass 10,000 and 500,000 Da, heparin (H-3149), dimethyl sulfoxide (DMSO), salts and buffers were from Sigma (St. Louis, Mo.). 96-well polyvinylchloride microplates were from Costar (Cambridge, Mass., Cat. No. 2596). Anti-mouse IgG horseradish peroxidase-conjugate and HEPES were from ICN (Costa Mesa, Calif.). K-Blue peroxidase substrate was from Neogen (Louisville, Ky.). HiTrap recombinant protein A (r-pA) column (1 ml) was from Amersham Pharmacia Biotech (Piscataway, N.J.). Nucleohistone was from Worthington (Lakewood, N.J.).

Antibody Activity Determination.

The antibody binding to commercial nucleohistone preparation adsorbed to poly-L-lysine-coated plates has been done by enzyme-linked immunosorbent assay (ELISA) (Iakoubov, L. Z., et al., Oncol. Res. 9: 489-446 (1997)). To assess the effect of modification on antibody activity we used the the 2C5 monoclonal antibody concentration providing 20% or 30% of maximal response in each experiment, using non-modified 2C5 monoclonal antibody as 100% reference point (i.e. biological activity given for non-modified 2C5 monoclonal antibody is set at 100%).

Antibody Modification.

To 8.5 μl of 0.94 mg/ml 2C5 monoclonal antibody in 10 mM HEPES, pH 7.5 1 μl of DexSO₄ (10 mg/ml in the same buffer) or buffer, and 0.5 μl of SPDP (0.5 mM in absolute ethanol, freshly prepared) was sequentially added and the mixture was incubated for 1 hr at room temperature. Those experimental conditions correspond to results represented by solid squares in FIG. 2. The 2C5 monoclonal antibody modification was performed also in other conditions in order to have proper controls for interpretation of results. Results for the non-modified 2C5 monoclonal antibody control are represented by open circles of FIG. 2. This non-modified 2C5 monoclonal antibody control was taken through exactly the same modification procedures with the exception of addition of DTPA-A. Results of incubation of the 2C5 monoclonal antibody with dextran sulfate alone is represented by open squares of FIG. 2. Results of conjugation of the 2C5 monoclonal antibody with SPDP in the absence of a protective agent are represented by solid circles of FIG. 2. Unless otherwise mentioned, the other parameters of the reaction (i.e.: temperature, pH, molarity of buffers, time of incubation etc.) were exactly the same for each point of the graph.

Antibody activity was determined by ELISA. Briefly, after performing modifications of the antibody according to the conditions specified herein, the modified-antibody (or controls) was applied to plates coated with poly-L-lysine-and nucleohistone preparation. Serial dilutions of the antibody (or controls) were performed inside the plate in order to obtain the following concentration of antibody: 0.010, 0.032, 0.100, 0.316, 1.000, 3.162, 10.000 μg/ml). An Anti-mouse IgG horseradish peroxidase conjugated antibody was added. The plate was washed and the peroxidase substrate (K-blue) was added. The optical density in each wells of the plate was measured at 630 nm, following the enzymatic reaction. Raw data of the ELISA are presented in table 2 below. Row 1 of table 2 represents data obtained for the non-modified 2C5 monoclonal antibody control. Row 2 of table 2 represents data obtained for the 2C5 monoclonal antibody conjugated with SPDP in the absence of protecting agent. Row 3 of table 2 represents data obtained for the 2C5 monoclonal antibody incubated with dextran sulfate. Row 4 of table 2 represents data obtained for the 2C5 monoclonal antibody protected by dextran sulfate and conjugated with SPDP.

TABLE 2

| | ROW NUMBER | | | | [2C5] |
|---|---|---|---|---|---|
| LANE | 1 | 2 | 3 | 4 | (μg/ml) |
| A | 1.598 | 1.117 | 0.854 | 0.715 | 10.000 |
| B | 1.603 | 0.463 | 0.635 | 0.742 | 3.162 |
| C | 1.296 | 0.219 | 1.750 | 0.901 | 1.000 |
| D | 0.903 | 0.081 | 1.285 | 0.694 | 0.316 |
| E | 0.399 | 0.022 | 0.588 | 0.247 | 0.100 |
| F | 0.133 | 0.008 | 0.260 | 0.077 | 0.032 |
| G | 0.045 | 0.007 | 0.095 | 0.029 | 0.010 |
| SPDP (mM) | 0 | 0.5 | 0 | 0.5 | |
| Heparin (mg/ml) | 0 | 0 | 1 | 1 | |

To demonstrate generalization of the protection method an alternative modifying agent was employed. A widely used reagent which has the ability to acylate amino groups but does not bring any negative charge (such as SPDP) was employed. FIG. 2 illustrates that modification of the 2C5 monoclonal antibody with SPDP without protection of the active site also leads to the loss of the antigen binding activity of the antibody. (FIG. 2, solid circles compared to open circles). As illustrated in Example 2, dextran sulfate (10,000 Da) also enables the 2C5 monoclonal antibody the keep at least part of its antigen binding activity during modification with SPDP (FIG. 2, solid squares compared with open circles). Again, dextran sulfate alone has no impact on the 2C5 monoclonal antibody antigen binding activity (FIG. 2, open squares compared to open circles). These results illustrate the generalization of the protection method described herein.

### Example 3

### 2C5 Monoclonal Antibody Modification with DTPA-A in the Presence of Heparin (Data are Presented in FIG. 3)

Reagents.

The origin of the mouse hybridoma producing the 2C5 monoclonal antibody was described earlier (Iakoubov, L. Z., et al., Oncol. Res. 9: 489-446 (1997)). Hybridoma was grown as an ascites and the 2C5 monoclonal antibody was purified by ammonium sulfate precipitation (at 50% saturation) and subsequent ion-exchange chromatography on DEAE-Toyopearl 650M (Sigma, St. Louis, Mo.). Diethylenetriaminepentaacetic acid anhydride (DTPA-A), 3-(2-pyridyldithio)propionic acid N-hydroxysuccinimide ester (SPDP), dextran sulfate (DexSO₄), molecular mass 10,000 and 500,000 Da, heparin (H-3149), dimethyl sulfoxide (DMSO), salts and buffers were from Sigma (St. Louis, Mo.). 96-well polyvinylchloride microplates were from Costar (Cambridge, Mass., Cat. No. 2596). Anti-mouse IgG horseradish peroxidase-conjugate and HEPES were from ICN (Costa Mesa, Calif.). K-Blue peroxidase substrate was from Neogen (Louisville, Ky.). HiTrap recombinant protein A (r-pA) column (1 ml) was from Amersham Pharmacia Biotech (Piscataway, N.J.). Nucleohistone was from Worthington (Lakewood, N.J.).

Antibody Activity Determination.

The antibody binding to commercial nucleohistone preparation adsorbed to poly-L-lysine-coated plates has been done by enzyme-linked immunosorbent assay (ELISA) (Iakoubov, L. Z., et al., Oncol. Res. 9:489-446 (1997)). To assess the effect of modification on antibody activity we used the 2C5 monoclonal antibody concentration providing 20% or 30% of maximal response for each experiment, using non-modified 2C5 monoclonal antibody as 100% reference point (i.e. biological activity given for non-modified 2C5 monoclonal antibody is set at 100%).

Antibody Modification.

To 8.5 μl of 0.94 mg/ml 2C5 monoclonal antibody in 10 mM HEPES, pH 7.5 1 μl of heparin (10 mg/ml in the same buffer) or buffer, and 0.5 μl of DTPA-A (0.6-20 mM in DMSO, freshly prepared) was sequentially added and the mixture was incubated for 1 hr at room temperature. Those experimental conditions correspond to results represented by solid triangles in FIG. 3. The 2C5 monoclonal antibody modification was performed also in other conditions in order to have proper controls for interpretation of results. Results for the non-modified 2C5 monoclonal antibody-control are represented by the open circles of FIG. 3. The non-modified 2C5 monoclonal antibody control was taken through exactly the same modification procedures with the exception of addition of DTPA-A. Results of incubation of the 2C5 monoclonal antibody with heparin alone are represented by open triangles of FIG. 3. Results of conjugation of the 2C5 monoclonal antibody with DTPA-A alone are represented by solid circles of FIG. 3. Unless otherwise mentioned, the other parameters of the reaction (i.e.: temperature, pH, molarity of buffers, time of incubation etc.) were exactly the same for each point of the graph.

Antibody activity was determined by ELISA. Briefly, after performing modifications of the antibody according to the conditions specified herein, the modified-antibody (or controls) was applied to plates coated with poly-L-lysine-and nucleohistone preparation. Serial dilutions of the antibody (or controls) were performed inside the plate in order to obtain the following concentration of antibody: 0.010, 0.032, 0.100, 0.316, 1.000, 3.162, 10.000 μg/ml). An Anti-mouse IgG horseradish peroxidase conjugated antibody was added. The plate was washed and the peroxidase substrate (K-blue) was added. The optical density in each wells of the plate was measured at 630 nm, following the enzymatic reaction. Raw data of the ELISA are presented in table 3 below. Row 1 of Table 3 represents data obtained for the non-modified 2C5 monoclonal antibody control. Row 2 of Table 3 represents data obtained for the 2C5 monoclonal antibody conjugated with DTPA-A in the absence of protecting agent. Row 3 of table 3 represents data obtained for the 2C5 monoclonal antibody incubated with heparin. Row 4 of Table 3 represents data obtained for the 2C5 monoclonal antibody protected with heparin and conjugated with DTPA-A.

TABLE 3

| | ROW NUMBER | | | | [2C5] |
|---|---|---|---|---|---|
| LANE | 1 | 2 | 3 | 4 | (μg/ml) |
| A | 2.681 | 1.010 | 0.721 | 0.923 | 10.000 |
| B | 2.898 | 0.348 | 0.616 | 0.877 | 3.162 |
| C | 2.360 | 0.169 | 1.936 | 1.791 | 1.000 |
| D | 1.442 | 0.068 | 1.362 | 1.334 | 0.316 |
| E | 0.781 | 0.03 | 0.844 | 0.693 | 0.100 |
| F | 0.361 | 0.032 | 0.350 | 0.242 | 0.032 |
| G | 0.182 | 0.051 | 0.274 | 0.092 | 0.010 |
| | −DTPA-A | +DTPA-A | −DTPA-A | +DTPA-A | |
| | −Heparin | −Heparin | +Heparin | +Heparin | |

To further demonstrate generalization of the protection method an alternative negatively charged polymer (anionic oligosaccharide) was employed. Heparin shares properties with nucleosome in that it is also negatively charged. Thus heparin was tested for its ability to serve as a protecting agent upon modification with DTPA-A.

The impact of heparin on the biological activity of the 2C5 monoclonal antibody was tested. Results illustrated in FIG. 3 (FIG. 3, open triangle) indicate that overall, heparin itself does not lead to a significant loss of the 2C5 monoclonal antibody's activity when compared to control (FIG. 3, open circles). An experiment was performed where heparin was employed for protecting the 2C5 monoclonal antibody's antigen binding site during conjugation. Treatment of the 2C5 monoclonal antibody with DTPA-A in the presence of heparin results in the preservation of its biological activity (FIG. 3, solid triangle) when compared to control (FIG. 3, open circles). Results of FIG. 3 demonstrate the effectiveness of heparin in protecting the antibody from losing its biological activity upon conjugation with DTPA-A. These results illustrate again the generalization of the protection method described herein.

### Example 4

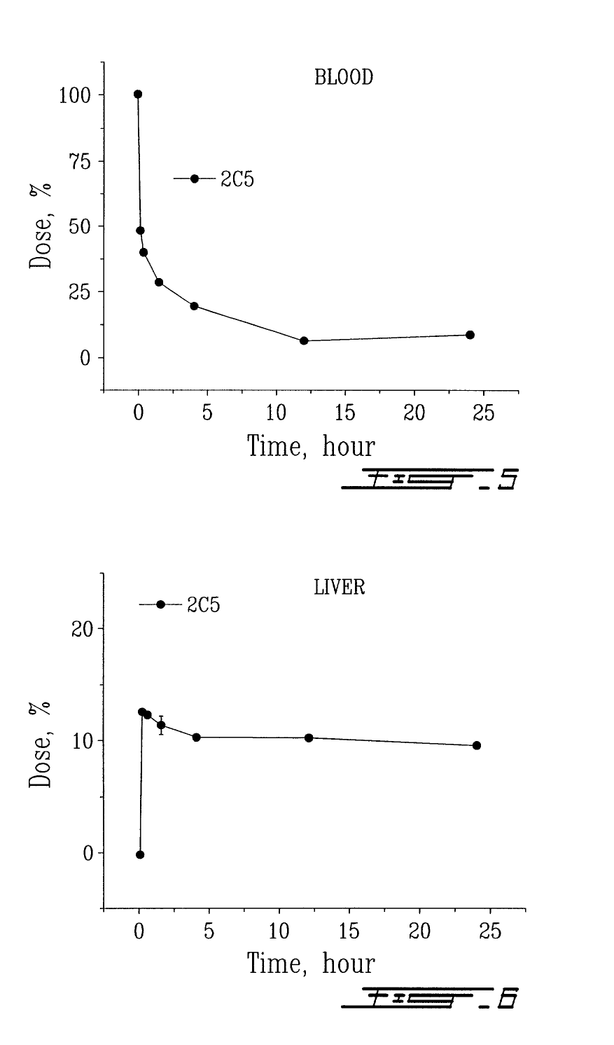

### Preparation of 2C5 ¹¹¹In DTPA-Conjugated Monoclonal Antibody and Demonstration of Specific Binding Activity. (Data are Presented in FIG. 4)

Reagents.

The origin of the mouse hybridoma producing the 2C5 monoclonal antibody was described earlier (Iakoubov, L. Z., et al., Oncol. Res. 9: 489-446 (1997)). Hybridoma was grown as an ascites and the 2C5 monoclonal antibody was purified by ammonium sulfate precipitation (at 50% saturation) and subsequent ion-exchange chromatography on DEAE-Toyopearl 650M (Sigma, St. Louis, Mo.). Diethylenetriaminepentaacetic acid anhydride (DTPA-A), 3-(2-pyridyldithio)propionic acid N-hydroxysuccinimide ester (SPDP), dextran sulfate (DexSO₄), molecular mass 10,000 and 500,000 Da, heparin (H-3149), dimethyl sulfoxide (DMSO), salts and buffers were from Sigma (St. Louis, Mo.). 96-well polyvinylchloride microplates were from Costar (Cambridge, Mass., Cat. No. 2596). HiTrap recombinant protein A (r-pA) column (1 ml) was from Amersham Pharmacia Biotech (Piscataway, N.J.). Nucleohistone was from Worthington (Lakewood, N.J.). ¹¹¹InCl₃ (397.5 Ci/mg) was from NEN Life Sciences Products (Boston, Mass).

¹¹¹In Labeling of 2C5 Monoclonal Antibody.

To 200 μl of 5.5 mg/ml 2C5 monoclonal antibody in phosphate buffered saline (PBS) 100 μl DexSO₄ in 10 mM HEPES, pH 7.5, 650 μl 10 mM HEPES, pH 7.5, and 50 μl DTPA-A (0.6-20 mM) in DMSO were added sequentially and the mixture was incubated for 1 hr at room temperature. The 250 μl of 5 M NaCl was added, the mixture was applied on the r-pA column equilibrated with 10 mM HEPES, pH 7.5, 1M NaCl. The column was washed with 10 ml of equilibration buffer at flow rate 0.4 ml/min and antibody was eluted with 0.1 M sodium citrate, pH 3.0. The antibody peak was collected and immediately neutralized by addition of one volume of 1 M Tris per four volumes of eluate. The neutralized mixture (about 1.5 ml) was dialyzed overnight at 4° C. against 1 l of 10 mM HEPES, pH 7.5, 150 mM NaCl and final antibody concentration was determined by measuring of absorbance at 280 nm (absorbance of 1.34 was used for 1 mg/ml mouse immunoglobulin solution). To 450 μl of this solution 50 μL of 1 M HEPES, pH 7.5 was added to prevent pH shift by the subsequent addition of acidic ¹¹¹InCl₃ solution. Then 4 μl of 0.1 M sodium citrate, pH 3.1 and 3 μl of ¹¹¹InCl₃ (about 30 μCi) in the same buffer was added and the sample was incubated for 1 hr at room temperature. Finally, it was dialyzed against 6 l of 10 mM HEPES, pH 7.5, 150 mM NaCl overnight at 4° C. The aliquots were withdrawn before and after dialysis. The radioactivity of aliquots were used to calculate the incorporation yield and specific radioactivity of preparation.

Determination of the Activity of the 2C5 ¹¹¹In DTPA Conjugated Monoclonal Antibody.

The binding of 2C5 ¹¹¹In DTPA-conjugated monoclonal antibody to nucleohistone preparation adsorbed to poly-L-lysine-coated plates has been done as for ELISA, but after the first incubation and washing the separate wells were cut out of the plate and counted in gamma-counter. Briefly, after performing modifications of the antibody according to the conditions specified herein, the modified-antibody was applied to plates coated with poly-L-lysine-and nucleohistone preparation. Serial dilutions were performed inside the plate in order to obtained the following concentration of antibody: 0.010, 0.032, 0.100, 0.316, 1.000, 3.162, 10.000 μg/ml). Wells from the plate were counted for radioactivity (expressed in counts per minute (cpm)), corresponding to the 2C5 ¹¹¹In DTPA-conjugated monoclonal antibody bound to the antigen. When needed the values were corrected for the decay of the isotope. The background count corresponding to the empty tube in the counter was subtracted from the obtained values. Results of binding of the 2C5 ¹¹¹In DTPA conjugated monoclonal antibody to the antigen-coated plates, are expressed as a function of 2C5 ¹¹¹In DTPA-conjugated antibody concentration. The binding activity of the 2C5 monoclonal antibody conjugated with DTPA-A and subsequently labeled with ¹¹¹In to nucleohistone preparation is illustrated in FIG. 4 (solid circles). The specific radioactivity (expressed in c.p.m.) remaining bound to plates after adsorption of the 2C5 ¹¹¹In DTPA-conjugated antibody and subsequent washing was significantly higher than the negative control (i.e. 2C5 ¹¹¹In DTPA-conjugated antibody bound to non-coated plates) (FIG. 4, open circles). Results of FIG. 4 indicate that the method used herein is useful to generate a biologically active antibody conjugates labeled with a radioactive isotope. Such reagents may be used in various ways such as in immunoscintigraphy using the biologically active antibody conjugate described in example 4 or in other types of assays such as immunofluorescence, radioimmunoassays, in vitro assay or for the targeted delivery of pharmaceutical agents when other types of modifying agent are used in the conjugation process.

### Example 5

### Pharmacokinetics of the 2C5 ¹¹¹In DTPA-Conjugated Monoclonal Antibody in Mice. (Data are Presented in FIGS. 5 to 9 and in Table 4)

Reagents.

Solution of 10 mg/ml 2C5 monoclonal antibody was prepared fresh in 10 mg/ml Dextran sulfate (10 000 Da) in 10 mM HEPES. The solution obtained was immediately 4-fold diluted with 10 mM HEPES. A 0.5 ml aliquot of 3.5 mg/ml DTPA-A in DMSO was added to 4 ml of 2C5 monoclonal antibody soution in Dextran sulfate. The 2C5 monoclonal antibody solution was continuously vortexed during DTPA-A addition. Resultant mixture was incubated at room temperature for 1 h. After incubation, 0.25 ml of 5M NaCl was added to the mixture.

The sample obtained was purified on a r-pA column pre-equilibrated with 10 mM HEPES, 1M NaCl, pH 7.4. The column was washed with around 10 volumes of binding buffer (10 mM HEPES, 1M NaCl, pH 7.4). The bound antibody was eluted with 0.1M sodium citrate, pH 3.0. Fractions of 0.3 ml each were collected. Sodium citrate was neutralized by addition of 750 μl of 1M Tris pH 8.0 to each fraction. Fractions containing proteins selected for an absorbance (A) of greater than 0.05 at a wave length of 280 nanometer (A₂₈₀), were pooled and dialized against 500-fold excess of HBS (HEPES-buffered saline). Dialized sample was concentrated 4-fold using an Amicon filter with 100,000 Da cut-off size. Preservation of DTPA-A-modified 2C5 monoclonal antibody activity was checked by ELISA. Conjugation with DTPA-A and labeling of the antibody with ¹¹¹In was performed as described in example 4.

Pharmakokinetics of the 2C5 ¹¹¹InDTPA-conjugated monoclonal antibody, was studied using CD-1 male mice weighting between 19 to 21 g. Each mouse received 100 μl of 0.9 mg/ml antibody via tail vein. Results for each time point were obtained on a group of 4 mice. At time points indicated in FIGS. 5 to 9 (i.e.: 0.167, 0.5, 1.5, 4, 12, 24 hour) mice were sacrificed by cervical dislocation.

The radioactivity present in each of the organ and tissue presented in FIGS. 5 to 9 was caused by the presence of the 2C5 ¹¹¹InDTPA-conjugated monoclonal antibody in these organs or tissues. The amount of antibody in these various organs was determined by radioactivity counting. The radioactivity associated with the initital dose of ¹¹¹In-labeled 2C5 monoclonal antibody given to the mice was given the value of 100%. The radioactivity associated with each organ and tissue was compared to the radioactivity associated with the initial dose.

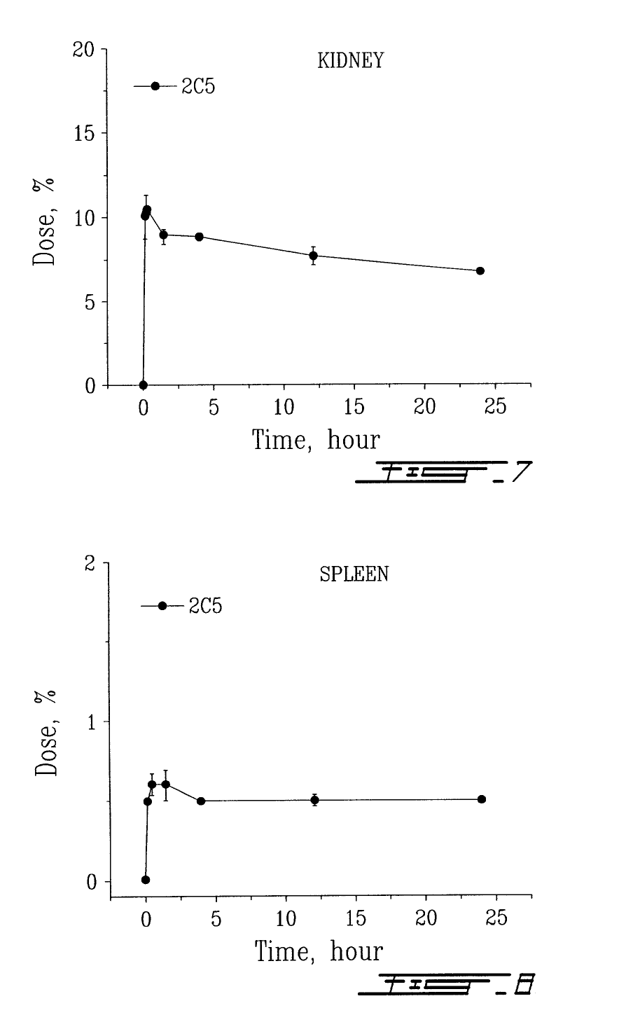

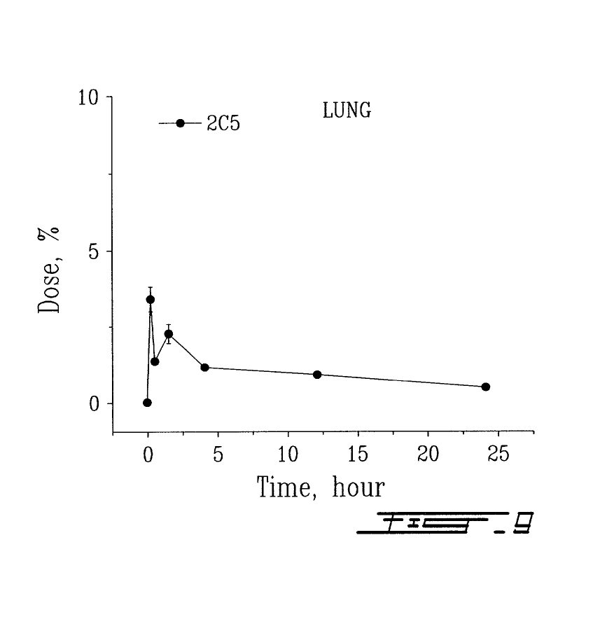

Results of FIGS. 5 to 9 are expressed as the percentage of the initial dose given to the animal that is found in each organ or tissue as a function of time. FIG. 5 represent the percentage of the initial dose found in blood, FIG. 6 represent the percentage of the initial dose found in the liver. FIG. 7 represent the percentage of the initial dose found in kidney. FIG. 8 represent the percentage of the initial dose found in the spleen. FIG. 9 represent the percentage of the initial dose found in the lung. A summary of the results presented in FIGS. 5 to 9 is also presented in table 4. Table 4 gives also results of the percentage of the initial dose found per gram of skin, the percentage of the initial dose found per gram of muscle as well as the percentage of the initial dose found per gram of blood, the percentage of the initial dose found per gram of kidney, the percentage of the initial dose found per gram of liver, the percentage of the initial dose found per gram of spleen, and the percentage of the initial dose found per gram of lung. Skin samples were obtained from mouse ears; muscle samples were taken form quadriceps.

Results illustrated in FIGS. 5 to 9 and table 4, indicates that the antibody conjugate described herein remains biologically active in vivo (i.e. inside an organism) and is useful to follow its fate inside the different body part of an organism. The use of a 2C5 monoclonal antibody conjugate is not restricted to animals. It may be used for example in immunoscintigraphic experiments in humans.

When an antibody, such as the 2C5 monoclonal antibody, selectively recognize cancer cells, biologically active 2C5 monoclonal antibody conjugate generated using the method described herein may be used as diagnostic and therapeutic tools. For example, a biologically active 2C5 monoclonal antibody conjugate may be used to monitor the presence of cancer cells in an organism and may be used for the targeted delivery of drugs (e.g. toxins, anticancer drugs).

TABLE 4

| | TIME POST INJECTION (hour) | | | | | |
|---|---|---|---|---|---|---|
| | 0.167 | 0.5 | 1.5 | 4 | 12 | 24 |
| % DOSE/g | 17.5 ± 0.3 | 14.5 ± 0.5 | 10.3 ± 0.8 | 7.2 ± 0.3 | 2.4 ± 0.6 | 3.2 ± 0.2 |
| blood 2C5 | | | | | | |
| % DOSE/g | 47.7 ± 0.9 | 39.5 ± 1.5 | 28.2 ± 2.0 | 19.5 ± 0.7 | 6.4 ± 1.5 | 8.6 ± 0.5 |
| blood 2C5 | | | | | | |
| % DOSE/g | 14.4 ± 1.1 | 16.1 ± 0.8 | 15.3 ± 0.7 | 14.4 ± 1.5 | 10.5 ± 0.7 | 11.6 ± 0.4 |
| kidney 2C5 | | | | | | |
| % DOSE/g | 10.4 ± 1.3 | 10.2 ± 0.9 | 8.9 ± 0.5 | 8.8 ± 0.2 | 7.7 ± 0.5 | 6.7 ± 0.2 |
| kidney 2C5 | | | | | | |
| % DOSE/g | 5.5 ± 0.2 | 5.7 ± 0.2 | 5.2 ± 0.3 | 4.6 ± 0.2 | 3.9 ± 0.3 | 4.0 ± 0.2 |
| liver 2C5 | | | | | | |
| % DOSE/g | 12.5 ± 0.3 | 12.2 ± 0.6 | 11.3 ± 0.9 | 10.2 ± 0.4 | 10.2 ± 0.4 | 9.6 ± 0.3 |
| liver 2C5 | | | | | | |
| % DOSE/g | 4.7 ± 0.4 | 4.7 ± 0.4 | 5.2 ± 1.4 | 3.5 ± 0.1 | 3.7 ± 0.3 | 3.4 ± 0.2 |
| spleen 2C5 | | | | | | |
| % DOSE/g | 0.5 ± 0.1 | 0.6 ± 0.1 | 0.6 ± 0.1 | 0.5 ± 0.1 | 0.5 ± 0.1 | 0.5 ± 0.1 |
| spleen 2C5 | | | | | | |
| % DOSE/g | 12.2 ± 1.0 | 6.5 ± 0.3 | 8.2 ± 0.9 | 4.1 ± 0.8 | 3.6 ± 0.1 | 2.3 ± 0.1 |
| lung 2C5 | | | | | | |
| % DOSE/g | 3.3 ± 0.4 | 1.4 ± 0.1 | 2.3 ± 0.3 | 1.2 ± 0.1 | 0.9 ± 0.1 | 0.5 ± 0.4 |
| lung 2C5 | | | | | | |
| % DOSE/g | 6.4 ± 0.9 | 5.7 ± 0.6 | 5.1 ± 1.1 | 4.6 ± 2.0 | 5.1 ± 0.5 | 5.9 ± 0.7 |
| skin 2C5 | | | | | | |
| % DOSE/g | 1.3 ± 0.1 | 1.2 ± 0.1 | 1.1 ± 0.1 | 1.0 ± 0.2 | 1.3 ± 0.1 | 1.9 ± 0.3 |
| muscle 2C5 | | | | | | |

The inventors demonstrate the production of an antibody protein conjugate (¹¹¹In-labeled 2C5 monoclonal antibody protein) as a final product that may be used in potential immunoscintigraphic experiments. The examples showed herein demonstrate a method to protect an antibody by protective agents (such as dextran sulfate), other than the antigen to enable the protein to keep at least part of its activity prior or during modification.

This example demonstrates also a method to dissociate the modified antibody from the protective agent (e.g., dextran sulfate) after modification. To achieve dissociation and purification of the antibody conjugate, the inventors used the approach based on the results of antibody modification at high ionic strength.

As shown in FIG. 1 (closed triangles), 1 M NaCl abolishes the protective ability of dextran sulfate considerably, presumably by inducing the dissociation of the 2C5 monoclonal antibody-dextran sulfate complex. Therefore, we used affinity chromatography on recombinant protein A (r-pA) column at high ionic strength (1 M NaCl) to dissociate dextran sulfate from the antibody bound to the column. The washing of the column with ten column volumes of high ionic strength buffer removed both dextran sulfate and hydrolyzed DTPA-A. The antibody was then eluted by pH 3.0 buffer, dialyzed and labeled with ¹¹¹In (i.e., a reporter molecule). The results of the labeling of the 2C5 monoclonal antibody with ¹¹¹In and modified with different concentrations of DTPA-A are presented in Table 5.

Table 5 illustrates that the use of lower concentration of DTPA-A leads to the lower degree of ¹¹¹In incorporation and consequently to the lower specific radioactivity of final preparation probably due to the lower degree of modification. However, using higher ratio ¹¹¹In: 2C5-DTPA-conjugated monoclonal antibody, the inventors could achieve higher specific radioactivity, up to 400 μCi per milligram of antibody conjugate (e.g. using lower amount of antibody at labeling step). To test the antibody activity of the final preparation we studied the binding of ¹¹¹In-labeled 2C5 monoclonal antibody to the plate coated with antigen (nucleohistone) or uncoated. The plot shown as FIG. 4 shows that the antibody conjugate labeled with ¹¹¹In is able of specific binding to the antigen and that essentially no binding is observed in wells that does not contain the antigen. The data in FIG. 1 reveals the results pertaining to the preparation of highest specific radioactivity from Table 5, but other preparations presented in Table 5 show a similar pattern of binding with the maximal binding values ranging from 1,100 -2,700 cpm.

TABLE 5

| [DTPA-A] | ¹¹¹In | Specific Activity |
|---|---|---|
| (μM) | Incorporation (%) | (μCi/mg) |
| 31 | 30.6 | 24.5 |
| 62 | 46.5 | 47.5 |
| 125 | 51.8 | 43.0 |
| 250 | 72.1 | 68.3 |
| 500 | 79.6 | 92.7 |

The new methods may be used to protect a variety of biologically active proteins such as antibodies, including active fragments (i.e. Fc, F(ab)2, F(ab)2′ and Fab) and humanized antibodies, enzymes, receptors, cytokines, chemokines, growth factors, hormones and transcription factors, against loss of their biological activity prior or during subsequent modification or conjugation, such as chemical modifications (e.g., modification with DTPA-A or SPDP).

The protective group described herein may, as described above, be a negatively charged polymer (natural and synthetic polymers) such as carboxymethyl-cellulose, carboxymethyl-starch and carboxymethyl-dextran, anionic polysaccharides and anionic oligosaccharides. Anionic polysaccharides and anionic oligosaccharides comprise, for example, dextran sulfate (DexSO₄) and heparin (Hep). The protective groups described herein may be added prior or during the modification process, depending upon the nature of the protective groups and the modifying agent added by conjugation.

Modifying agent that may be added to the biologically active proteins also vary and may include pharmaceutical agents, solid supports or substrates, reporter molecule, groups carrying a reporter molecule, acylating agents, chelating agents, cross-linking agent, targeting groups, and ligand/binding groups. Reporter molecules include fluorescent molecules, enzymes (such as horseradish peroxidase, alkaline phosphatase), dyes, radioactive atoms and isotopes (e.g., indium, iodine and technetium), and superparamagnetic and paramagnetic agents such as gadolinium, iron and manganese.

Chelating agents include DTPA and EDTA. Cross-linking agent includes SPDP. Solid support includes liposome, colloidal gold, microparticles, or microcapsule. Ligands and binding groups include one of a pair of such ligand/binding groups such as biotin and avidin or streptavidin. Pharmaceutical agent includes a toxin, a drug, and a pro-drug. Targeting groups include antibody fragments, hormones and lectines. Acylating agents include DTPA-A. The modifying agent may be attached to the proteins via covalent or noncovalent or other types of bonds.

The new protein conjugates may be used for diagnostic or therapeutic purposes depending on the biologically active protein and the modifying agent moiety. For example, antibodies that bind specifically to a certain type of cancer, or to all types of cancers, such as the 2C5 monoclonal antibody, can be attached to cytotoxic agents (e.g., doxorubicin and cisplatin) to provide selectivity to cancer cells and targeted anticancer therapy.

### Other Embodiements

It is to be understood that while the invention has been described in conjunction with the detailed description thereof, that the foregoing description is intended to illustrate and not limit the scope of the invention, which is defined by the scope of the appended claims. Other aspects, advantages, and modifications are within the scope of the following claims.

We claim:

1. A method of preparing a biologically active protein conjugate, the method comprising:
combining a biologically active protein moiety with a protective group under conditions that allow the protective group to removably bind to the protein to provide a protected protein;
modifying the protected protein by the addition of a modifying agent moiety selected from the group consisting of a pharmaceutical agent, a solid support, a reporter group, and a targeting group; and
removing the protective group to provide a biologically active protein conjugate comprising a biologically active protein moiety and the modifying agent moiety.

2. The method of claim 1, wherein the protective group is a negatively charged polymer.

3. The method of claim 1, wherein the protective group is selected from the group consisting of dextran sulfate, heparin and mixtures thereof.

4. The method of claim 1, wherein the biologically active protein is a protein that binds to nucleic acids.

5. The method of claim 1, wherein the biologically active protein is an antibody, an enzyme, a receptor, a cytokine, a chemokine, a growth factor, a hormone, a transcription factor, a peptide, or a peptide analog.

6. The method of claim 1, wherein the modifying agent moiety is a solid support.

7. The method of claim 6 wherein the solid support is a liposome.

8. The method of claim 1, wherein the modifying agent moiety is a reporter molecule.

9. The method of claim 1, wherein the biologically active protein binds to a specific protein, glycoprotein, lipid, carbohydrate, or nucleic acid.

10. The method of claim 1, wherein the biologically active protein binds to a complex of one or more proteins, glycoproteins, lipids, carbohydrates, or nucleic acids.

11. A biologically active protein conjugate comprising a first moiety and a second moiety, wherein the first moiety is a biologically active protein and wherein the second moiety is selected from the group consisting of a pharmaceutical agent, a solid support, a reporter molecule, and a targeting group.

12. The conjugate of claim 11, further comprising a protective group.

13. The conjugate of claim 11, wherein the biologically active protein is selected form the group consisting of an antibody, an enzyme, a receptor, a cytokine, a chemokine, a growth factor, a hormone, a transcription factor, a peptide, and a peptide analog.

14. The conjugate of claim 11, wherein the biologically active protein binds to a specific protein, glycoprotein, lipid, carbohydrate, or nucleic acid.

15. The conjugate of claim 11, wherein the second moiety is a solid support.

16. The conjugate of claim 11, wherein the second moiety is a liposome.

17. The conjugate of claim 11, wherein the second moiety is a reporter molecule.

18. The conjugate of claim 12, wherein the protective group is selected from the group consisting of dextran sulfate, heparin and mixtures thereof.

19. A method of preparing a biologically active protein conjugate, the method comprising:
combining a biologically active protein with a protective group selected from the group consisting of anionic polysaccharides, anionic oligosaccharides, and mixtures thereof to provide a protected protein under conditions that allow the protective group to removably bind to the protein; and
modifying the protected protein by the addition of a modifying agent selected from the group consisting of a pharmaceutical agent, a solid support, a reporter molecule, a group carrying a reporter molecule, a chelating agent, an acylating agent, a cross-linking agent, and a targeting group.

20. The method of claim 19 further comprising the step of removing the protective group to provide a biologically active protein conjugate comprising a biologically active protein and a modifying agent.

21. The method of claim 20 wherein the step of removing the protective group is performed by contacting the protein conjugate with a high ionic strength solution.

22. The method of claim 19, wherein the protective group is selected from the group consisting of dextran sulfate, heparin and mixtures thereof.

23. The method of claim 19, wherein the biologically active protein is selected from the group consisting of an antibody, an enzyme, a receptor, a cytokine, a chemokine, a growth factor, a hormone, a transcription factor, a peptide, and a peptide analog.

24. The method of claim 19, wherein the biologically active protein is a protein that binds to nucleic acids.

25. The method of claim 19, wherein the biologically active protein binds to a specific protein, glycoprotein, carbohydrate, lipid, and nucleic acid.

26. The method of claim 19, wherein the biologically active protein binds to a complex of one or more proteins, glycoproteins, carbohydrates, lipids, and nucleic acids.

27. The method of claim 19, wherein the modifying agent is a solid support selected from the group consisting of carbohydrates, liposomes, lipids, colloidal gold, microparticles, microcapsules, microemulsions, and the matrix of an affinity column.

28. The method of claim 19, wherein the modifying agent is a reporter molecule selected from the group consisting of a fluorophore, a chromophore, a dye and an enzyme.

29. The method of claim 19 wherein the modifying agent is a group carrying a reporter molecule selected from the group consisting of chelating agents, and crosslinking agents.

30. A biologically active protein conjugate comprising a first moiety and a second moiety, the first moiety is a protected biologically active protein and the second moiety being selected from the group consisting of a pharmaceutical agent, a solid support, a reporter molecule, a group carrying a reporter molecule, a chelating agent, an acylating agent, a cross-linking agent, and a targeting group, the protected biologically active protein being associated with a protective group selected from the group consisting of anionic polysaccharides, anionic oligosaccharides and mixtures thereof.

31. The conjugate of claim 30, wherein the protected biologically active protein is an antibody, an enzyme, a cytokine, a chemokine, a growth factor, an hormone, a receptor, a transcription factor, a peptide, or a peptide analog.

32. The conjugate of claim 30, wherein the protected biologically active protein binds to a specific protein, glycoprotein, and nucleic acid.

33. The conjugate of claim 30, wherein the protected biologically active protein binds to a complex of one or more proteins, glycoproteins, carbohydrates, lipids, and nucleic acids.

34. The conjugate of claim 30, wherein the second moiety is a solid support selected from the group consisting of carbohydrates, liposomes, lipids, colloidal gold, microparticles, microcapsules, microemulsions, and the matrix of an affinity column.

35. The conjugate of claim 30, wherein the second moiety is a reporter molecule selected from the group consisting of a fluorophore, a chromophore, a dye and an enzyme.

36. The conjugate of claim 30, wherein the second moiety is a group carrying a reporter molecule selected from the group consisting of a chelating agent, and a cross-linking agent.

37. The conjugate of claim 30, wherein the protective group is selected from the group consisting of dextran sulfate, heparin and mixtures thereof.

38. The method of claim 1, wherein the modifying agent moiety is a lipid.

39. The method of claim 1, wherein the modifying agent moiety is a carbohydrate.

40. The method of claim 1, wherein the modifying agent moiety is a microemulsion.

41. The conjugate of claim 11, wherein the second moiety is a lipid.

42. The conjugate of claim 11, wherein the second moiety is a carbohydrate.

43. The conjugate of claim 11, wherein the second moiety is a microemulsion.
